(19) United States
(12) Patent Application Publication
(10) Pub. No.: US 2008/0178872 A1
(43) Pub. Date: Jul. 31, 2008

(54) DOSE SELECTIVE BREATH ACTUATED INHALER

(76) Inventors: Perry Genova, Chapel Hill, NC (US); Robert C. Williams, Raleigh, NC (US); Prashant P. Kakade, Cary, NC (US); Robin Hyder, Morrisville, NC (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/947,019

(22) Filed: Nov. 29, 2007

Related U.S. Application Data
(60) Provisional application No. 60/868,152, filed on Dec. 1, 2006.

(51) Int. Cl. A61M 15/00

(52) U.S. Cl. 128/200.23

(57) ABSTRACT

A dose selective breath actuated inhaler including a meterless canister storing a pressurized medicament, and a vacuum actuated release, where application of a vacuum to the inhaler initiates a release of the medicament in the canister. The inhaler includes a mechanism for dialing a proper dose of pressurized medicament, a computer for generating a plurality of signals including a solenoid trigger signal, and a solenoid which upon receipt of a solenoid trigger signal actuates a solenoid arm to end the release of the medicament from the canister.

### CROSS-REFERENCE SECTION TO RELATED APPLICATIONS

This application claims the benefit of U.S. Provisional Patent Application No. 60/868,152, filed Dec. 1, 2006, which is hereby incorporated by reference in its entirety.

### FIELD OF THE INVENTION

The present invention relates to a metered dose inhaler and in particular to a metered dose inhaler that uses a variable timer for administration of desired amounts of drug from a meterless aerosol canister depending upon a patient's need.

### BACKGROUND OF THE INVENTION

In an effort to provide for a non-invasive means of administering insulin and other systemic drugs to patients, and thereby eliminate the need for syringes, aerosolized formulations have been theorized.

Heretofore, the studies and experiments in the pulmonary delivery of insulin have suffered from poor reproducibility of the dose to be inhaled. Typically, known inhaler devices utilize a metered dose canister that dispenses a discrete quantity of drug each time the valve is depressed. Known dry powder devices, for example, typically employ small reservoirs for each drug dose whereby the reservoirs are individually emptied into the inhaled air stream at each actuation. Dry powder inhalers are also generally less precise and robust as compared to liquid metered dose inhalers. Importantly, both dry powder inhalers and metered dose inhalers for the delivery of liquids provide only a specific dose amount. Users that require more of a drug than is available in one dose may be required to go through the actuation sequence multiple times in order to receive the proper amount of drug. Due to the limitation on dosage amount in these known inhalation devices, a user may not even be able to dose properly according to the precise amount needed.

Aside from diabetes and its treatment with insulin, a number of other diseases require the active participation and understanding of the patient to provide for accurate, and therefore, effective dosing. Examples of such diseases are chronic obstructive pulmonary disease (COPD), asthma, and other respiratory problems.

Thus, there is a need for a device and method providing for the effective and variable dosing for a patient to insure that effective amounts of drug are received at the desired time. There is a further need that such a device is user-friendly providing adequate administration of the drug preferably in a single inhalation.

The present invention endeavors to overcome the problems of the prior art and provide a non-invasive device and methodology for delivery of drugs that produces repeatable and variable/controlled dosage amounts of a drug to the patient substantially without the need for complex circuitry having high-energy demands.

### SUMMARY OF THE INVENTION

One aspect of the instant invention is directed to a dose selective breath actuated inhaler including a meterless canister storing a pressurized medicament, and a vacuum actuated release, where application of a vacuum to the inhaler initiates a release of the medicament in the canister. The inhaler also includes a computer for generating a plurality of signals including a solenoid trigger signal, and a solenoid that upon receipt of a solenoid trigger signal actuates a solenoid arm to end the release of the medicament from the canister.

According to another aspect of the present invention, a dose selection device is used with a metered dose inhaler that enables a user to dial in the appropriate dose and thereafter initiate release of the medicament, inhaling until the user is signaled to stop inhaling (e.g., when the selected dose has been fully administered).

Another aspect of the instant invention is drawn to a method of administering a substance to a human patient by inhalation including providing an inhaler with a meterless canister containing the substance, selecting a dose of the substance by manipulating a dose-selector on the inhaler, and inhaling the selected dose.

Still another aspect of the instant invention is a method of administering a substance including steps of providing an inhaler including a meterless canister storing a pressurized medicament, a vacuum actuated release, a computer for generating a plurality of signals including a solenoid trigger signal, and a solenoid having a solenoid arm. The method also includes steps of applying a vacuum to a mouthpiece portion of the inhaler to trigger release of the medicament in the canister, running a clock function from the time release of the medicament begins, and generating a solenoid signal at the completion of the clock function, the solenoid signal actuating the solenoid arm to cease release of the medicament from the canister.

Other embodiments of the present invention will be described in greater detail below.

### BRIEF DESCRIPTION OF THE DRAWINGS

Further characteristics, features, and advantages of the present invention will be apparent upon consideration of the following detailed description of the invention taken in conjunction with the following drawings, and in which:

FIG. 1 is a perspective view of an inhaler according to one aspect of the present invention;

FIG. 2 is a perspective view of the inhaler of FIG. 1 in the cocked position;

FIG. 3 is a perspective view of the inhaler of FIG. 1 showing its two-piece construction with the insertion of a drug canister;

FIG. 4 is a cross-sectional view of an inhaler according to one aspect of the present invention in the stored position;

FIG. 5 is a cross-sectional view of an inhaler according to one aspect of the present invention in the cocked position;

FIG. 6 is a cross-sectional view of an inhaler according to one aspect of the present invention at the point when the user begins to inhale;

FIG. 7 is a cross-sectional view of an inhaler according to one aspect of the present invention during administration of the drug;

FIG. 8 is a cross-sectional view of an inhaler according to one aspect of the present invention following administration of the drug;

FIG. 9 is a cross-sectional view of an inhaler according to one aspect of the present invention following administration and closure of the cover to return the device to its stored position; and

FIG. 10 is a schematic diagram of an electrical circuit according to one aspect of the instant invention.

### DETAILED DESCRIPTION

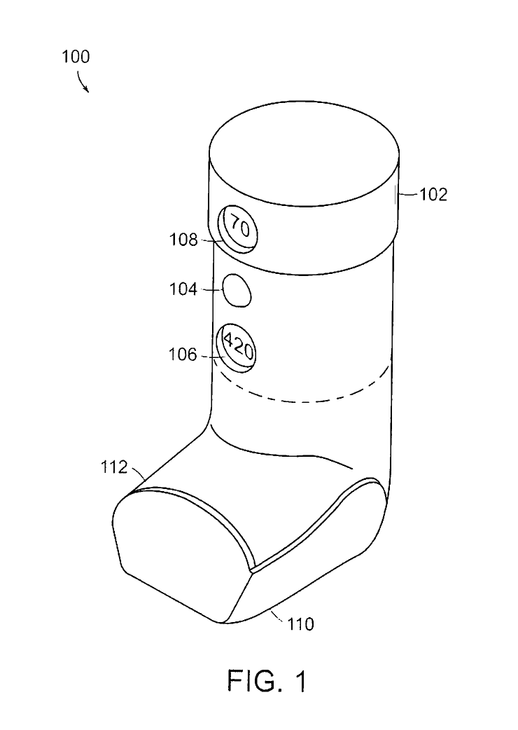

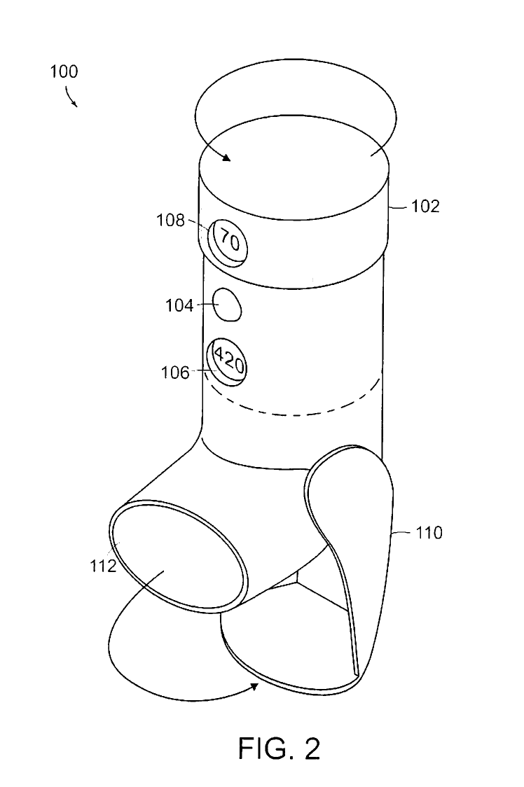

FIG. 1 shows a dose selective breath actuated inhaler according to one aspect of the present invention. The dose selective inhaler 100 includes a dial 102 which may be turned by the user to alter the dose to be administered by the inhaler 100. The dose selective inhaler 100 also includes a (lose administration indicator, for example, a LED (light-emitting diode) inhalation light 104, which indicates to the user when the medication is being dispensed. The inhalation light 104 is just one example of a structure that may be used to indicate to a user when the user has begun to inhale and when to stop inhaling in order to receive the proper dose. Other dose administration indicators, whether visual, auditory, or tactile (e.g., a vibrating device, which may be important for a blind patient) may be used. The inhaler 100 is a breath-actuated inhaler where the patient's inhalation triggers the medication release. The inhalation light 104 turns on upon cocking of the device as shown in the progression from FIG. 1 to FIG. 2, and continues to be illuminated as the device administers a dose, turning off once the dose has been fully administered to signal to the user that they can cease inhaling. The breath-actuated inhaler 100 of the present invention is described in detail below. Importantly, other devices that incorporate a meterless canister and contain the necessary connectivity between a dose selection dial 102, a computer 126, and an inhalation light 104 or other means for indicating to the patient to continue or discontinue inhaling while the dose is being administered, can be used to administer drugs using the dose selection technology of the present invention.

Referring again to FIG. 1, the inhaler 100 includes a units-remaining indicator 106. As can be readily understood by those of skill in the art, in a device that has variable dosing characteristics, accounting for the volume of medicament already administered and the amount remaining in the device is important so that the patient is never in a situation where there are no doses remaining when they are in need of the medication. In practice, doses of medication are not counted, (as in the traditional dose counters) rather International Units or IU's are counted. As a result, when a patient determines that they require a dose of 70 IU's as shown in FIGS. 1 and 2, the user twists the dial 102 until the dose-setting indicator 108 reads 70. The dose-setting indicator 108 may be an LCD, but may also be a simple printed indicator, which is uncovered as the dial 102 is rotated. The user then rotates the cover 110 to expose the mouthpiece 112. At this position, the inhaler 100 is ready to administer a dose of 70 IU's to the patient. Upon administration of the 70 IU's, the value will be subtracted from the number of IU's indicated as remaining in the inhaler by units-remaining display 106.

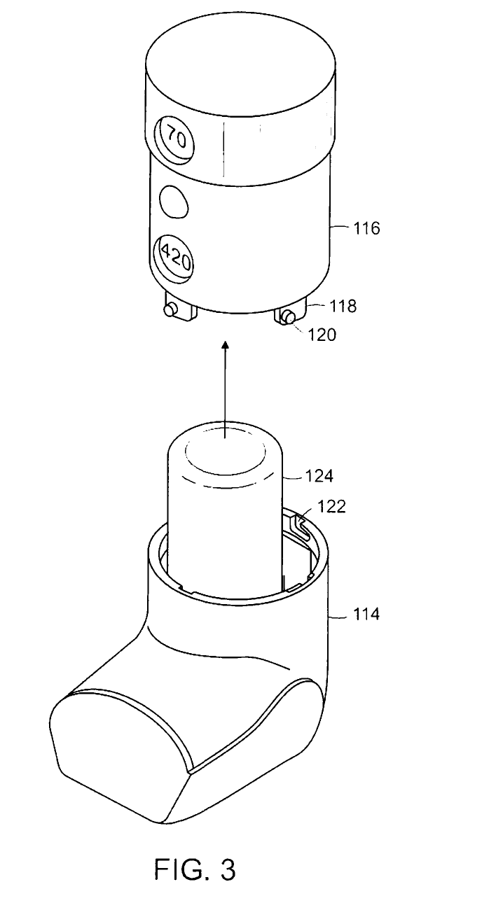

FIG. 3 shows the inhaler 100 opened into its two component parts: a base 114 and a cap 116. The cap 116 can be joined to the base 114 by any suitable means known to those of skill in the art. As shown in FIG. 3, tabs 118 are formed on the cap 116 and include projections 120 extending outwardly therefrom. The projections 120 fit into slots 122 formed in the base 114. The slots 122 may, as shown, have an L-shape allowing the cap 116 to be rotated into the slots 122 and secured to the base 114. Situated in the base 114 is a canister 124, preferably a meter-less canister, which is a canister that, upon actuation, will continue to dispense the pressurized medication contained therein until either the entire canister is emptied, or the pressure causing the actuation of the canister is released.

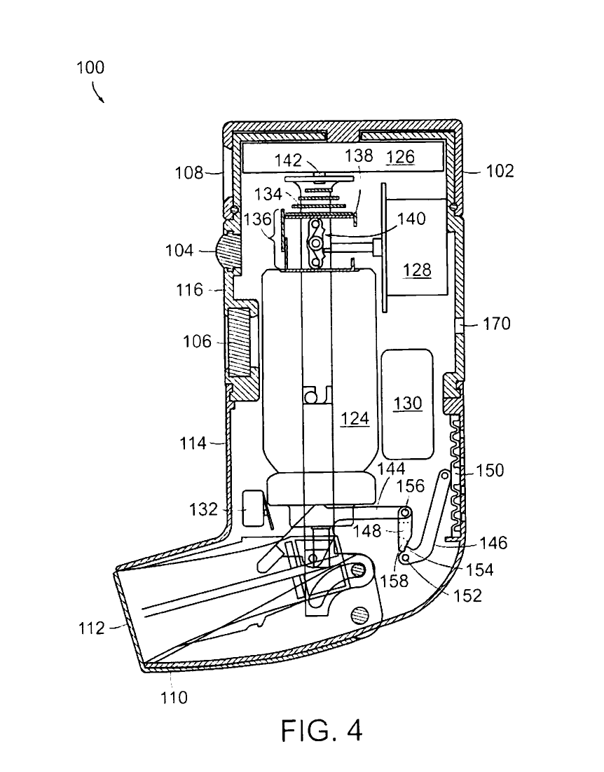

FIG. 4 shows a cross sectional view of a dose selective breath actuated inhaler 100 according to one aspect of the instant invention. The inhaler 100 includes a meterless canister 124. As shown in FIG. 4 the inhaler 100 is in the stored or at-rest position. The mouthpiece 112 is covered by a hinged cover 110, which, as will be discussed, also acts as the cocking mechanism for the inhaler 100.

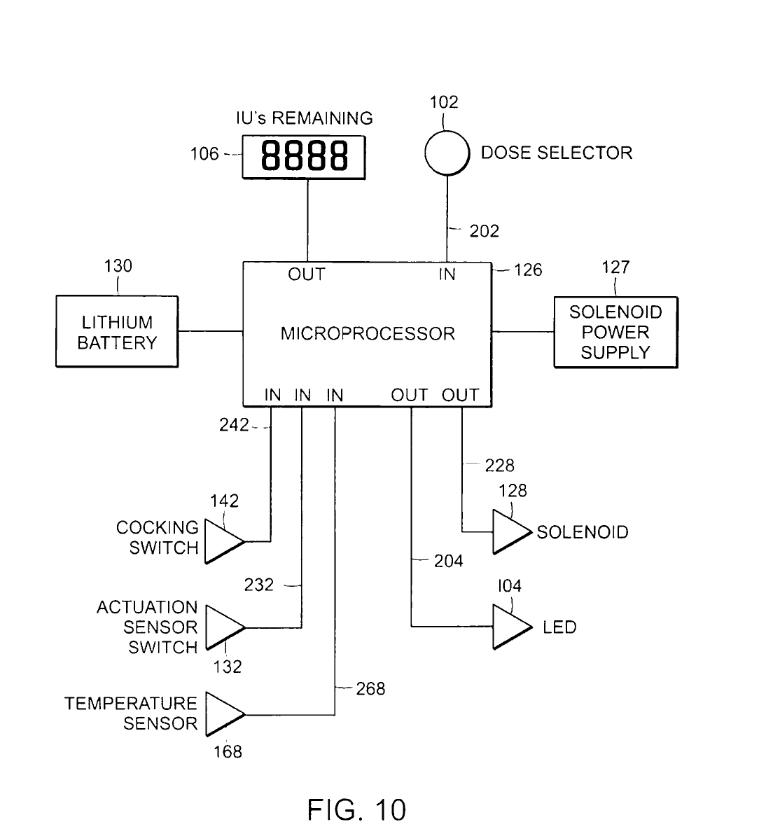

The inhaler 100 shown in FIG. 4 also includes a timing and display control or computer 126. The computer 126 is electrically connected to a solenoid 128 and a battery 130, as well as the units-remaining display 106, the inhalation light 104, an actuation sensor switch 132, a cocking switch 142, and preferably the dose-setting indicator 108. The computer and its interconnection to various components are outlined in detail below with respect to FIG. 10.

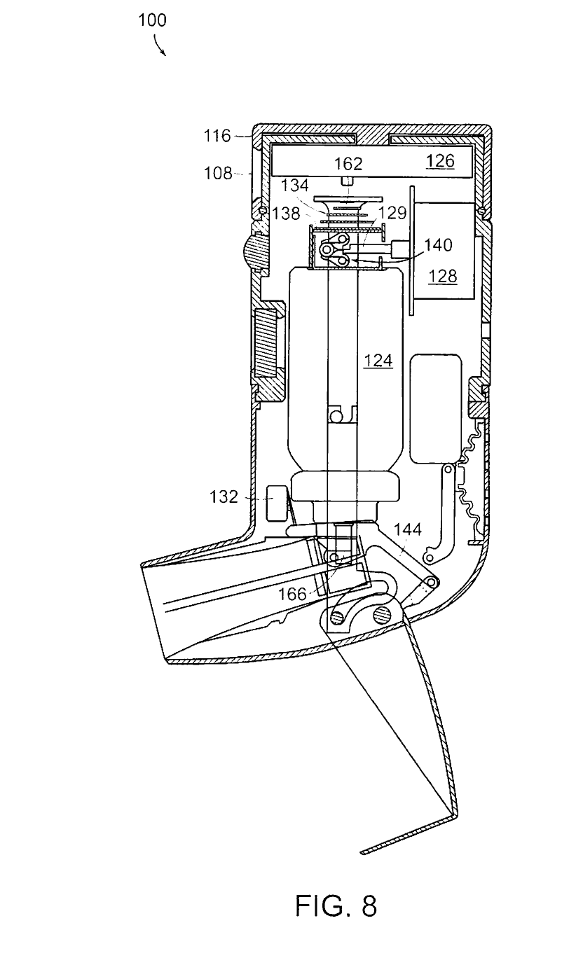

The cap 116 contains a spring 134, which rests in a spring release mechanism 136. The spring release mechanism rests on the canister 124. The spring release mechanism 136 includes two concentric cups 138 that are in vertical sliding engagement with one another and a spring biased collapsible knuckle 140. The spring biased collapsible knuckle 140 prevents the two cups 138 from collapsing into each other under the pressure applied by the spring 134 when in its extended position as shown in FIG. 4. Upon application of pressure in a direction perpendicular to the longitudinal dimension of the spring biased knuckle 140, the spring biased knuckle collapses, as shown in FIG. 8. The collapse of the spring biased knuckle 140 will be described in greater detail below.

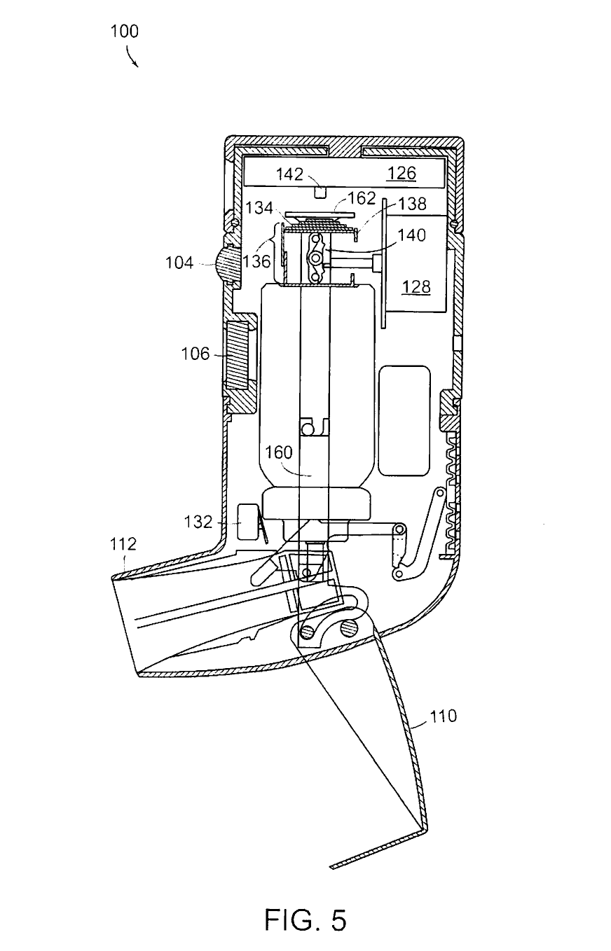

The cap 116 also contains the cocking switch 142 that provides an electrical signal to the computer 126 when it senses that the inhaler has moved from the at-rest position shown in FIG. 4 to the cocked position shown in FIG. 5. When the inhaler 100 is in its at-rest position, the switch 142 is depressed. When the inhaler 100 is moved to the cocked position as shown in FIG. 5, the switch 142 is released. A further feature of the cap 116 is an orifice 170 that allows for the entry of air from the atmosphere into the inhaler 100 which assists in dispersing the medicament and providing a volume to be inhaled with the medicament by the user.

The cap 116 includes a dial 102. The dial 102 is connected either mechanically, or preferably electrically to computer 126. By this connection, rotation of the dial 102 alerts the computer 126 of the size of the dose, that is, the number of IU's to be administered. The computer 126 calculates a time period for dose administration based on the spray rate of the canister 124. As a result the user is able to adjust the size of a medicament dose and the device indicates to the user, via the inhalation light 104, how long to continue inhaling until the full dose has been administered.

The inhaler 100 includes a release mechanism that includes a rocker 144, a cam 146, a follower 148, and a diaphragm 150. In FIG. 4, the cam 146 is connected on one end to the diaphragm 150, and is held in place in the inhaler 100 by a pin 152 on the other end. The cam 146 also includes a lip 154 which is formed on the end of the cam 146 connected to the pin 152. The follower 148 is connected to the rocker 144 by another pin 156; the follower 148 is free to rotate about the pin 156. In addition, the follower 148 has a lip 158 which interacts with the lip 154 as bill be discussed below.

As mentioned above, FIG. 4 shows the inhaler 100 in the at-rest position. In this position the spring 134 is in a less biased state and the cups 138 are spread apart from one another by the spring biased knuckle 140. The cocking switch 142 is depressed and the follower 148 rests on the cam 146.

As shown in FIG. 5, the cover 112 is open exposing the mouthpiece 110. The movement of the cover 112 causes a rod 160 having a head 162 to compress the spring 134 against the top cup 138. The movement of the head 162 also releases the cocking switch 142. The release of the cocking switch 142 sends a signal to the computer 126 indicating that the inhaler is now cocked and ready to administer drug. In one preferred embodiment it is the release of the cocking switch 142 that signals the computer 126 to change from a sleep-mode to an on-mode. In the sleep-mode, in a preferred embodiment, only the units-remaining display 106 is illuminated and drawing electrical power. Upon entering the on-mode the computer 126 begins to function, calculating the time for actuation of the inhaler based on the dialed-in dose and the ambient temperature within the housing, the solenoid 128 is powered, the inhalation light 104 is powered, and the inhalation switch 132 is monitored. As described herein, the cocking switch 142 is a normally closed switch, that when the pressure of the spring is removed therefrom closes the electrical circuit connecting the cocking switch 142 to the computer 126.

In accordance with another embodiment of the present invention, the computer 126 can be turned on to the on-mode by manual depression of a switch (not shown) on the exterior of inhaler 100.

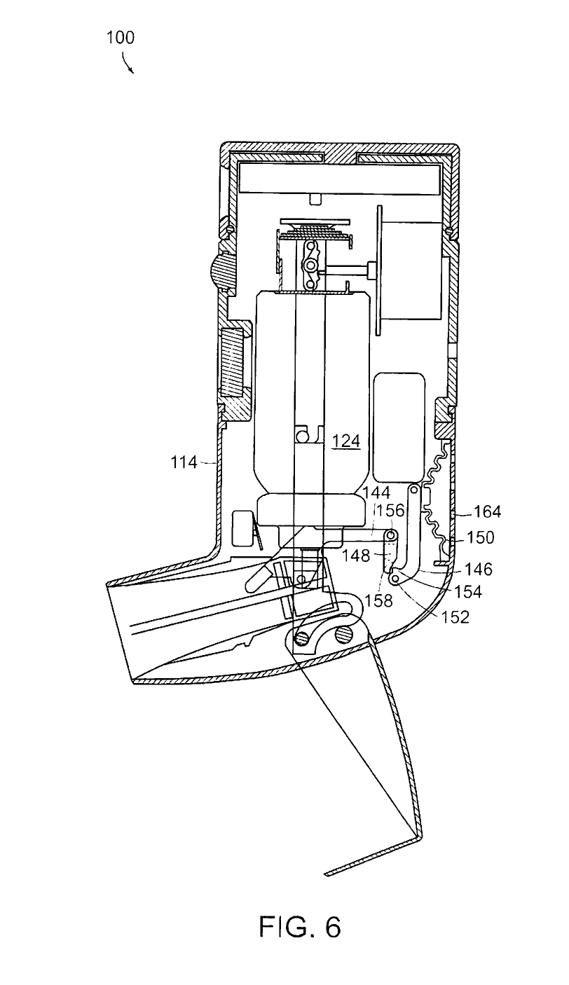

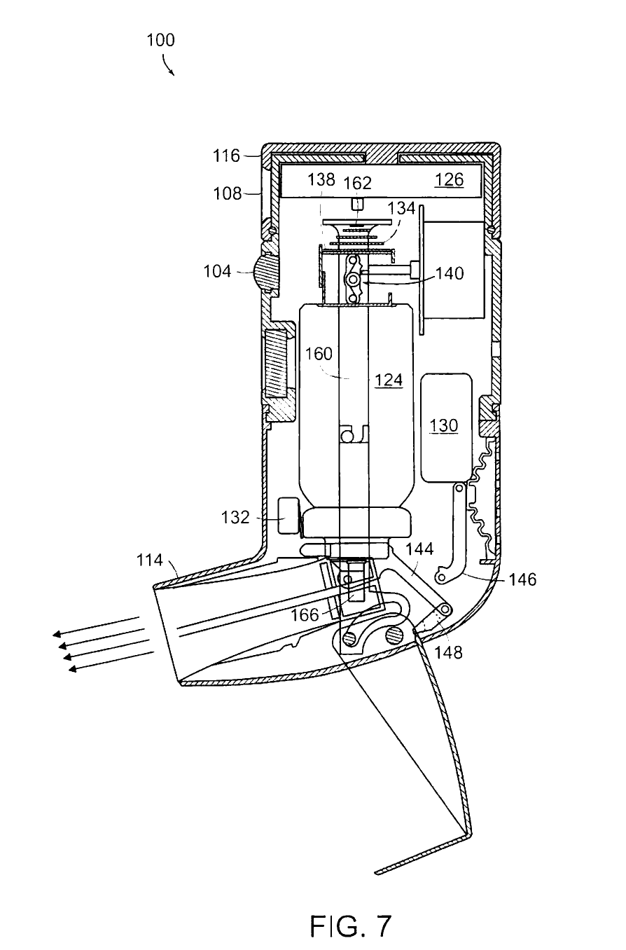

In FIG. 6, the patient begins to inhale. The patient's inhalation causes a vacuum in the interior of the inhaler. This vacuum causes the diaphragm 150 to deform in the direction of the user's mouth, which is the origin of the vacuum. The deformation of the diaphragm 150 is assisted by holes 164 formed in the base 114 which allow air to enter the base 114 of the inhaler 100 on a backside of the diaphragm 150. The air that enters the inhaler at the back side of the base 114 is of a higher pressure than the vacuum created internally in the inhaler 100, which thereby causes the deformation of the diaphragm 150. The movement of the diaphragm 150 causes movement of the cam 146. e.g., rotation, about the pin 152. The movement or the cam, 146 causes the lip 154 formed on the cam 146 to put pressure oil the lip 158 of the follower 148. The follower 148 begins to rotate about the pin 156 connected to the rocker 144 because the rocker is held in place by the canister 124. As the diaphragm 150 continues to expand, lip 154 formed on the cam 146 forces the follower 148 off of the cam 146 as shown in FIG. 7.

Upon release of the follower 148 from the cam 146, the rocker 144 is free to pivot. With respect to FIG. 7 the rotation is in a clock-wise direction. The movement of the rocker 144 releases the canister 124 and allows the spring 134 to expand forcing the canister 124 to move in the direction of the base 114. The canister 124 includes a spring biased stem 166 connected to a valve (not shown) in the canister 124, initially, the spring pressure asserted by the spring 134 overcomes the spring internal to the canister 124, causing the valve to open and release the pressurized medicament from the canister.

The expansion of the spring 134 is enabled by the release of the rocker 144. The spring 134 acts on the top cup 138 on one side and against the head 162 of the rod 160. The head 162 of the rod 160 prevents the expansion of the spring 134 in the direction of the cap 116. Because the cups 138 are prevented from collapsing by the spring-biased knuckle 140, the spring 134 causes the canister 124 to move downward releasing the medicament as described above. In addition, this movement triggers the actuation sensor switch 132. This triggering sends a signal to the computer indicating that dispensing of medicament has begun. The spring 134, as shown in FIG. 7, is in a less biased position than as shown in FIG. 5 or 6. The closure of the actuator sensor switch 132 sends a signal to the computer 126 to begin running of a clock function within the computer 126. The clock function as will be described below continues sending power to illuminate the inhalation light 104 for a time specified depending on, for example, the dose to be administered. Upon the running of the clock for the time specified, the inhalation light 104 will turn off signaling to the user that they can stop inhaling, as the complete dose has been administered by the inhaler 100. Other functions of the actuator sensor switch 132 and the computer 126 are discussed below with respect to FIG. 10.

FIG. 8 shows the inhaler 100 following completion of dispensing a dose to a user. As described above with respect to FIG. 7, upon movement of the canister 124 downward by the release of the rocker 144, the canister 124 closes the actuation sensor switch 132 and begins to dispense medicament. This triggering of the actuation sensor switch 132 sends a signal to the computer 126 to run a clock function. The duration of the clock function is calculated by the computer 126 based on the number of IU's to be administered and the spray rate of the canister 124. Upon the completion of clock function, the computer 126 sends a signal to the solenoid 128. This signal causes the solenoid 128 to extend the solenoid arm 129 and collapse the spring biased knuckle 140, as shown in FIG. 8. The solenoid arm 129 accesses the spring-biased knuckle 140 via slots (not shown) in one side of the cups 138. This collapsing of the knuckle 140 allows the pressure of the spring 134 to collapse the slidingly engaged cups 138. As a result, the spring 134 extends in the vertical direction. At a predetermined extension offspring 134, the spring force generated by the spring 134 is less than the spring force of the internal spring in the canister 124 which acts on the spring biased stem 166. When the spring force of the internal spring (not shown) in the canister 124 is greater than the force of spring 134, the canister 124 moves vertically upward, the internal canister valve (not shown) closes, and administration of the dose ends. As described above, in the preferred embodiment the inhaler 100 uses a meterless canister, which permits dispensation from the inhaler 100 of as little or as great of a dose as required by the user.

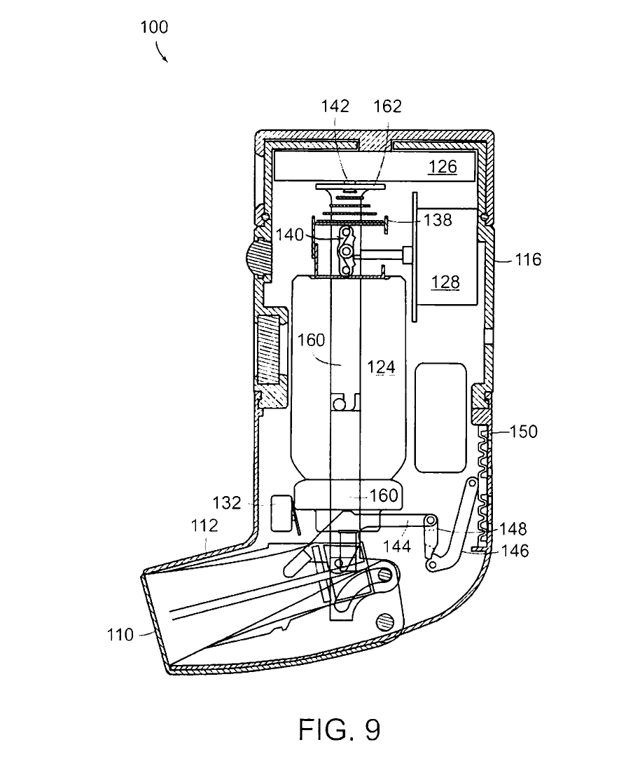

FIG. 9 shows the return of the inhaler 100 to the at-rest position after completion of dose dispensation to a user. The cover 112 is closed covering the mouthpiece 110. The movement of the cover 112 acts on the rod 160 to move the canister 124 in the direction of the cap 116. The movement of the rod 160 releases some of the spring pressure created by the spring 134 and allows the spring in the spring biased knuckle 140 to return the knuckle to its extended position which simultaneously causes the cups 138 to move distally away from each other. The head 162 contacts the cocking switch 142 and depresses it sending a signal to the computer 126. The rocker 144 is returned to its at-rest position, which in turn returns the follower 148 onto the cam 146, which has also returned to its at-rest position once the pressure inside the inhaler and the pressure out side the inhaler have equalized following administration of the dose. This may be assisted by making the diaphragm 150 of a material that is biased in the direction away from cam 146. Preferably, the diaphragm is made of an elastomeric material. The movement of the canister 124 also removes the pressure applied to the actuator sensor switch 132. Either the release of the actuator sensor switch, or the depression of the cocking switch can be used to return the computer 126 to a sleep mode. Alternatively, the computer can be shut off by pressing an “off” button (not shown) on the inhaler 100 exterior or by releasing an “on” button (not shown) on the inhaler 100 exterior.

The computer 126 will now be discussed with respect to FIG. 10. The computer 126 is connected to the battery 130, which provides electrical power for the inhaler 100, and a solenoid power supply 127 which supplies power to the solenoid 128. The battery 130 also supplies power to the electrical components of the inhaler 100 including the inhalation light 104, the units-remaining display 106, the dose-setting indicator 108, and the switches, as well as the computer 126. As will be understood by one skilled in the art, the computer 126 can be programmed to receive numerous inputs and perform various functions.

First, according to one embodiment of the present invention, the computer 126 receives input from the dose selector 102. By rotating the dose selector 102, an electrical contact (not shown) on the cap 116 is contacted by an electrical contact (not shown) in the dial of the dose selector 102 forming a circuit. The parameters of this circuit create the dose selector signal 202 which is supplied to the computer 126 and is used to determine the time period for a clock signal. As will be discussed below, the clock signal is used to provide the time period for illuminating the inhalation light 104 following triggering of the inhaler 100 to release a dose, as well as the time period for supplying power to the solenoid 128 that ultimately ceases the dose administration. This dose signal 202 also determines the number of IU's to be deducted from the units-remaining display 106.

Another input received by the computer 126 is the cocking switch input 242, which is received once the inhaler has been cocked as shown in FIG. 5. In one embodiment this may be a normally closed switch, that when the pressure applied by the head 162 of the rod 160 removed, returns to its closed position to complete the circuit. This may be, for example, to act as an on/off switch for the inhaler 100 so that power is conserved at all times except when the device is cocked and ready to administer the drug. As a result, when the inhaler 100 is returned to the at-rest position as shown in FIG. 9, following administration of the drug, the power to the inhaler is shut off except for the display of the IU's remaining 106, which is preferably constantly maintained.

The computer 126 also receives an actuation sensor signal 232 from the actuation sensor switch 1332. When a user inhales on the mouthpiece 110, the release of a dose is triggered (as discussed above), which causes the canister 124 to move in the direction of the base 114 and close the normally open actuation sensor switch 132. The closure of this switch sends a signal to the computer 126 to start a clock signal that illuminates the inhalation light 104 by supplying a signal 204. Other means of indicating to the user how long the user must inhale in order to receive the proper dose (i.e. other dose administration indicators, whether visual, auditory, or tactic) may also be used, in which case the computer 126 is programmed so that the clock signal triggers the dose administration indicator. Prior to the user's inhalation, the computer 126 has performed a calculation based on the dose selector signal 202. Once the actuation sensor switch 132 is switched on, the inhalation light 104 will be turned on signaling to the user to continue inhaling. Upon expiry or the running of the clock to zero, the computer 126 opens the circuit to the inhalation light 104 extinguishing the light and signaling to the user to stop inhaling. At the same time, signal 228 is sent to the solenoid 128 to actuate the solenoid arm 129 to stop the release of the medicament.

The actuation signal 232 also triggers calculation of the number of IU's being dosed, and the computer 126 deducts that amount from the units-remaining display 106. Alternatively, the computer 126 can be configured to deduct the amount of units-remaining from the display 106 upon closure of the mouthpiece cover, which returns the inhaler 100 to its at-rest position as shown in FIG. 9.

The computer 126 also receives an input from a temperature sensor 168 that provides a temperature signal 168. As will be appreciated by those of skill in the art, the temperature of the inhaler, which is generally near ambient, will affect the dispensation of a pressurized medicament. The higher the temperature, the higher the pressure that will be developed by the expansion of the propellant inside the canister 124, which affects the timing of release of the medicament, because medicament at a higher pressure will release a greater volume in a set period of time than when it is at a lower pressure. Accordingly, temperature may advantageously be factored into the calculation of the clock function of the computer 126.

Advantageously, as discussed above, the computer enables the user to set their desired dose and then have the inhaler indicate the number of IU's remaining after each administration so that each patient can manage medication usage and receive the proper amount of medication dependent upon the patient's needs. As will be understood by one of ordinary skill in the art, the computer 126 of the present invention can be configured and programmed to perform a wide range of functions not limited to the functions described herein.

Another aspect of the present invention is the use of the dosage triggering and timing mechanism described above in a breath actuated nasal drug delivery device as described in commonly assigned and co-pending U.S. patent application Ser. Nos. 11/160,493 and 11/418,527, the contents of which are incorporated herein by reference.

The type of medicament used therein does not limit the present invention. Examples of drugs that can be used with the present invention are short-acting β2-agonists such as albuterol and salbutamol, which provide quick relief from acute asthma symptoms. Long-acting β2-agonists such as salmeterol and formoterol are used to control asthma symptoms over a longer period of time. Another class of drugs contemplated in the present invention are anticholinergics such as ipratropium bromide, which helps prevent bronchospasms in COPD patients. Corticosteroids, such as budesonide, fluticasone and triamcinolone acetonide, are often used in asthma treatment for their anti-inflammatory effects. The instant invention can be used to deliver any of these drugs, as well as any combination thereof, such as for example, flutiform, a combination of fluticasone (a corticosteroid) and formoterol.

Typically active ingredients in the formulations used in an inhaler as shown in FIG. 1 are readily made as suspensions or solutions with highly volatile propellants, such as for example. HFA-134(a) or HFA-227. Common pressurized aerosolized formulations well known in the industry are contemplated in this invention, and it will be understood by one of skill in the art that this includes formulations containing various excipients and stabilizers, such as for example, oleic acid, aspartame, water, ethanol, ethanoic acid, phosphatidyl choline, etc.

While certain formulations and diseases have been specifically discussed herein, the present invention is not so limited and may be used with any formulation deliverable with a metered dose inhaler.

Thus by the foregoing examples, the objects and advantages of the present invention are realized, and although preferred embodiments have been disclosed and described in detail herein, its scope and objects should not be limited thereby; rather its scope should be determined by that of the appended claims.

What is claimed is:

1. A dose selective breath actuated inhaler comprising:
a meterless canister storing a pressurized medicament,
a vacuum actuated release for triggering release of the medicament in the canister;
a computer for generating a plurality of signals including a solenoid trigger signal; and
a solenoid comprising a solenoid arm, which upon receipt of the solenoid trigger signal, actuates the solenoid arm to end the release of the medicament from the canister.

2. The dose selective breath actuated inhaler of claim 1 further comprising a dose selection dial for selecting a dose and generating a dose selector signal.

3. The dose selective breath actuated inhaler of claim 2, wherein said computer generates a clock function based on said dose selector signal and said clock function sets the amount of time a user must inhale on the inhaler in order to receive the dose selected.

4. The dose selective breath actuated inhaler of claim 1 further comprising a spring biased knuckle, wherein the solenoid arm acts on the spring biased knuckle upon receipt of the solenoid trigger signal to collapse the spring biased knuckle.

5. The dose selective breath actuated inhaler of claim 4, further comprising a spring and a pair of slidably engaged cups, wherein the spring acts on one of the cups, and the knuckle acts on both of the cups.

6. The dose selective breath actuated inhaler of claim 1 further comprising a cocking switch, said cocking switch which sends a signal to the computer to switch the inhaler from a sleep mode to an on mode when released and return the inhaler to a sleep mode when depressed.

7. The dose selective breath actuated inhaler of claim 3 further comprising an actuation sensor switch, wherein the actuation sensor switch when depressed sends a signal to the computer to begin the running of the clock function.

8. The dose selective breath actuated inhaler of claim 1 further comprising a units-remaining indicator denoting the number of doses remaining in the canister, said units-remaining indicator being updated by the computer following each actuation of the canister.

9. The dose selective breath actuated inhaler of claim 7 further comprising a dose administration indicator, wherein said dose administration indicator is turned on and off by the computer based on the start and stop of the clock function.

10. The dose selective breath actuated inhaler of claim 1, wherein the vacuum actuated release comprises:
a diaphragm mounted on a side wall of the inhaler;
a cam rotatably connected to the diaphragm on a first end and rotatably connected to a pin on a second end, said cam having a lip;
a follower rotatably connected to a rocker on one end and slidingly engaging the cam on the second end, said follower having a lip for engaging the lip of the cam;
said rocker engaging the canister at a first position, wherein upon inhalation on the inhaler, the diaphragm is deformed into the inhaler causing the cam to rotate and release the follower, which allows the rocker to rotate, with the rotation of the rocker allowing a spring force to move the canister into a second position and which releases the medication.

11. A dose selective breath actuated inhaler comprising:
a meterless canister storing a pressurized medicament;
a vacuum actuated release comprising a flexible diaphragm mounted in the wall of the inhaler, wherein application of a vacuum to the inhaler initiates a release of the medicament in the canister;
a computer for generating a plurality of signals including a solenoid trigger signal; and
a solenoid comprising a solenoid arm, which upon receipt of a solenoid trigger signal, actuates a solenoid arm to end the release of the medicament from the canister.

12. A dose selective inhaler comprising:
a meterless canister storing a pressurized medicament;
a dose selection dial for selecting the dose of medicament to be delivered from the canister;
a computer connected to said dose selection dial for determining the amount of medicament to be delivered from the canister based on the dose selected; and
a dose administration indicator connected to said computer for indicating that the medicament dose is being delivered.

13. The inhaler of claim 12 wherein the dose selection dial is mounted on the exterior of the inhaler.

14. The inhaler of claim 12 wherein the dose selection dial provides a signal to the computer for determining a clock function, which determines the amount of time that the dose administration indicator provides an indication to the user, and the dose administration indicator indicates to a user when to start and when to stop inhaling.

15. A method of administering a substance to a human patient by inhalation comprising:
providing an inhaler with a meterless canister storing the substance.
selecting a dose of the substance by manipulation of a dose-selector located on the inhaler, and
inhaling the selected dose.

16. The method of claim 15 wherein the step of inhaling the selected dose creates a vacuum in the mouthpiece portion of the inhaler, which triggers release of the substance in the canister.

17. The method of claim 15 wherein the step of selecting a dose of the substance is used by a computer to control a dose administration indicator which indicates to the user when to start and when to stop inhaling.

18. A method of administering a substance by inhalation comprising the steps of:
providing an inhaler including a meterless canister storing a pressurized medicament, a vacuum actuated release, a computer for generating a plurality of signals including a solenoid trigger signal, and a solenoid having a solenoid arm;
applying a vacuum to a mouthpiece portion of the inhaler to trigger release of the medicament in the canister;
running a clock function from the time release of the medicament begins;
generating the solenoid signal at the completion of the clock function, said solenoid signal actuating the solenoid arm to cease release of the medicament from the canister.

19. The method of claim 18, further comprising a step of cocking said inhaler.

20. The method of claim 18, wherein actuating the solenoid arm causes the collapse of a spring biased knuckle which ceases release of the medicament from the canister.

21. The method of claim 18, further comprising a step of dialing in a dose of medicament to be released from the canister, wherein the dialing in of the dose is used by the computer to determine the length of the clock function.

22. The method of claim 18, further comprising a step of indicating to the user when to start and when to stop inhaling and further comprising a step of updating a units-remaining indicator based on the amount of the medicament released.

23. The method of claim 22, wherein the dose of medicament released is calculated by the computer based on the dose dialed in.

24. The method of claim 18, wherein the application of a vacuum to the mouthpiece of the inhaler causes a diaphragm to deform and trigger the release of medicament from the canister.

25. The method of claim 18, further comprising a step of triggering an actuation sensor switch, wherein upon triggering of the actuation sensor switch, the clock function begins to run.
